SEQUENCE LISTING (11 sequences)

SEQ ID NO: 1 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 2 - protein, 5 aa, Artificial Sequence
  Synthetic sequence
SSIMH

SEQ ID NO: 3 - protein, 17 aa, Artificial Sequence
  Synthetic sequence
YIKPYNDGTKYNEKFKG

SEQ ID NO: 4 - protein, 12 aa, Artificial Sequence
  Synthetic sequence
EGGNDYYDTMDY

SEQ ID NO: 5 - protein, 11 aa, Artificial Sequence
  Synthetic sequence
RASQDINSYLS

SEQ ID NO: 6 - protein, 7 aa, Artificial Sequence
  Synthetic sequence
RVNRLVD

SEQ ID NO: 7 - protein, 9 aa, Artificial Sequence
  Synthetic sequence
LQYDAFPYT

SEQ ID NO: 8 - protein, 121 aa, Artificial Sequence
  Synthetic sequence
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 9 - protein, 108 aa, Artificial Sequence
  Synthetic sequence
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 10 - protein, 450 aa, Artificial Sequence
  Synthetic sequence
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 11 - protein, 214 aa, Artificial Sequence
  Synthetic sequence
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0393791 A1
(43) Pub. Date: Dec. 23, 2021

(54) COMBINATION TREATMENT WITH ANTI-CD123 ANTIBODY DRUG CONJUGATE AND PARP INHIBITOR

(71) Applicant: Health Research, Inc., Buffalo, NY (US)

(72) Inventors: Eunice Wang, Williamsville, NY (US); Scott Portwood, Orchard Park, NY (US)

(73) Assignee: Health Research, Inc., Buffalo, NY (US)

(21) Appl. No.: 17/289,519

(22) PCT Filed: Oct. 29, 2019
(86) PCT No.: PCT/US2019/058528

§ 371 Date: Apr. 28, 2021

Related U.S. Application Data
(60) Provisional application No. 62/753,468, filed on Oct. 31, 2018.

(51) Int. Cl. A61K 47/68; A61K 31/5517; A61K 31/502; A61K 31/5025; A61P 35/02

(52) U.S. Cl. CPC A61K 47/6849; A61K 47/6803; A61P 35/02; A61K 31/502; A61K 31/5025; A61K 31/5517

(57) ABSTRACT

A composition including an anti-CD123 antibody-drug conjugate and a poly ADP ribose (PARP) inhibitor can be used in therapy or as a medicament. The composition including the anti-CD123 antibody-drug conjugate and the poly ADP ribose (PARP) inhibitor can be used for treating cancer or for inducing cancer cell death in a population of cancer cells. Hematological cancers such as acute myeloid leukemia (AML) can be treated with the composition. Administering an anti-CD123 antibody-drug conjugate and a poly ADP ribose (PARP) inhibitor is a method of treating cancer. Administering, to a population of cancer cells, an anti-CD123 antibody-drug conjugate and a poly ADP ribose (PARP) inhibitor is a method for inducing cancer cell death.

### CROSS-REFERENCE TO RELATED APPLICATIONS

This application claims priority to and the benefit of co-pending U.S. provisional patent application Ser. No. 62/753,468, entitled Combination Treatment with Anti-CD123 Antibody Drug Conjugate and PARP Inhibitor, filed Oct. 31, 2018, which is incorporated herein by reference in its entirety.

### STATEMENT REGARDING FEDERALLY SPONSORED RESEARCH OR DEVELOPMENT

This invention was made with government support under P30CA016056 awarded by the National Cancer Institute. The government has certain rights in the invention.

### 1. TECHNICAL FIELD

The present invention is directed to compositions and methods for treating cancer that involve the combination of an anti-CD123 antibody drug conjugate and a poly ADP ribose (PARP) inhibitor.

### 2. BACKGROUND

CD123 (IL-3 receptor alpha-chain) is a therapeutic target for hematological malignancies based on its high expression levels in acute myeloid leukemia (AML), blastic plasmacytoid dendritic cell neoplasm (BPDCN), and other cancers. An anti-CD123 antibody-drug conjugate (ADC) has been evaluated previously for potential treatment of relapsed/refractory CD123-positive hematological malignancies. However, increased efficacy of ADCs is still needed for treatment of relapsed or refractory malignancies and is of significant therapeutic interest.

Citation or identification of any reference in Section 2, or in any other section of this application, shall not be considered an admission that such reference is available as prior art to the present invention.

### 3. SUMMARY

A pharmaceutical composition is provided comprising:

an anti-CD123 antibody-drug conjugate; and

a poly ADP ribose (PARP) inhibitor.

In an embodiment of the pharmaceutical composition, the PARP inhibitor is a PARP-1 inhibitor. In another embodiment, the PARP inhibitor is a PARP-2 inhibitor.

In an embodiment of the pharmaceutical composition, the PARP inhibitor is selected from the group consisting of:

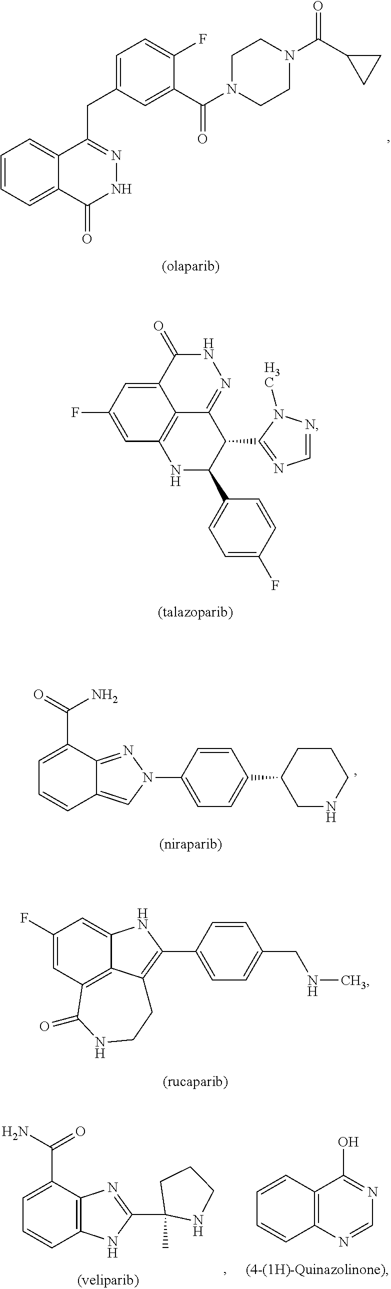

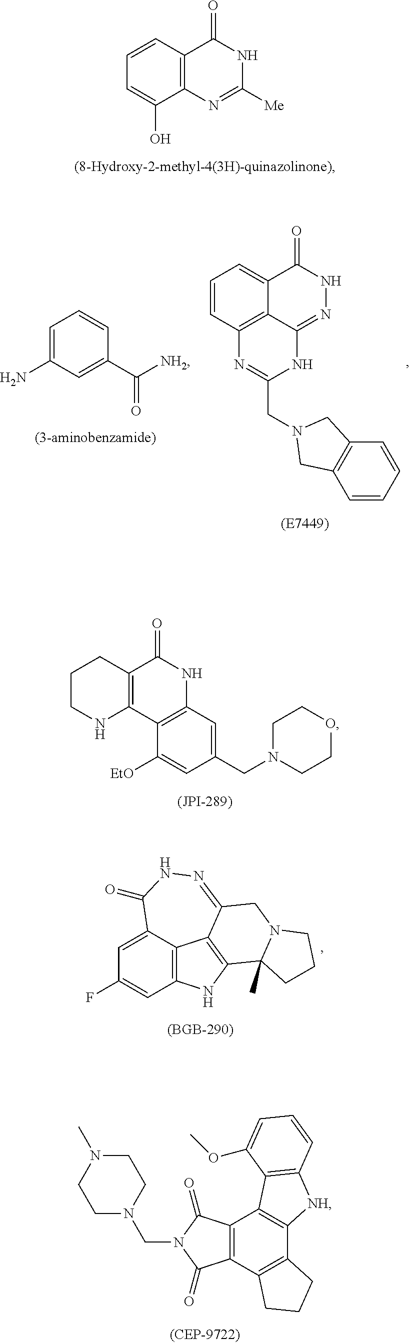 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

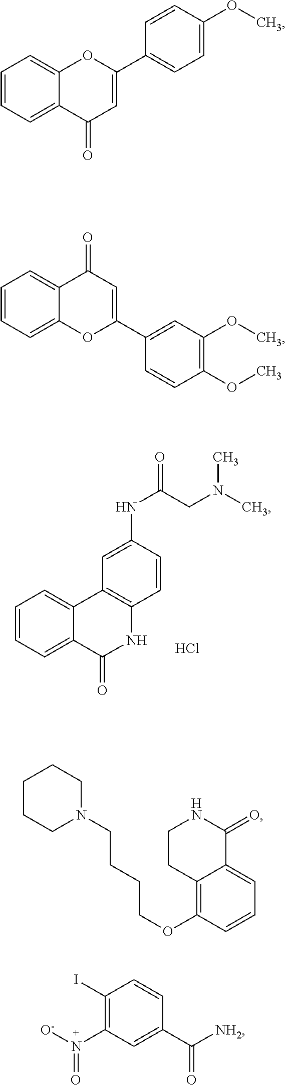

and a pharmaceutically acceptable salt thereof.

In another embodiment of the pharmaceutical composition, the PARP inhibitor is isolaparib or a pharmaceutically acceptable salt thereof.

In another embodiment of the pharmaceutical composition, the PARP inhibitor is talazoparib or a pharmaceutically acceptable salt thereof.

In another embodiment of the pharmaceutical composition, the anti-CD123 antibody-drug conjugate is an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, or an anti-CD123 antibody linked to camptothecin. In another embodiment, the indolinobenzodiazepine pseudodimer is IMGN632.

A method for treating cancer in a subject is also provided, the method comprising administering to the subject an anti-CD123 antibody-drug conjugate and a poly ADP ribose (PARP) inhibitor in an amount effective to treat the cancer.

In an embodiment of the method, the PARP inhibitor is a PARP-1 inhibitor. In another embodiment, the PARP inhibitor is a PARP-2 inhibitor.

In another embodiment of the method, the PARP inhibitor is selected from the group consisting of:

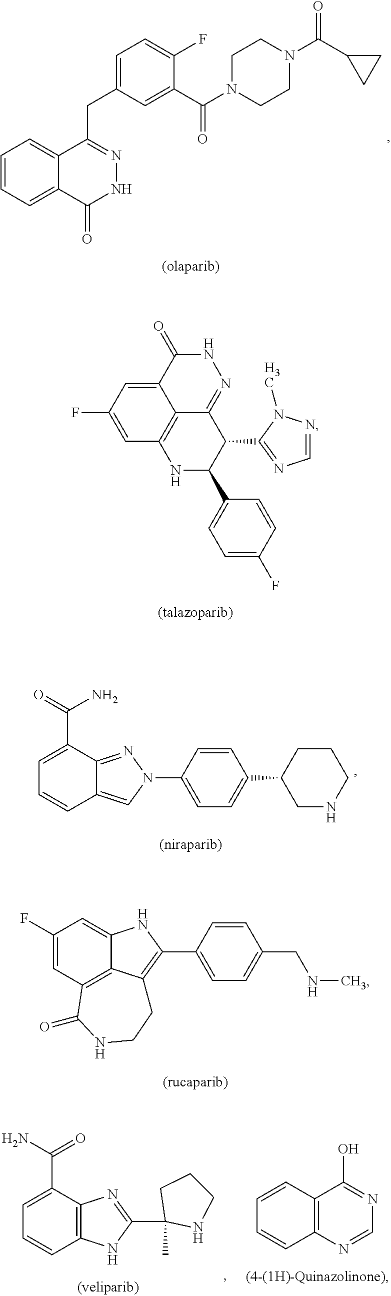

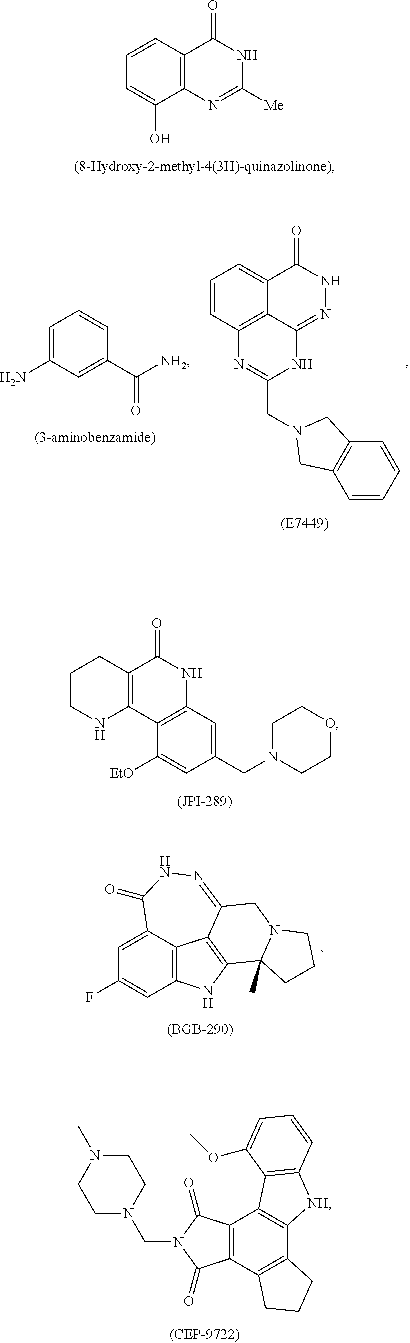 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

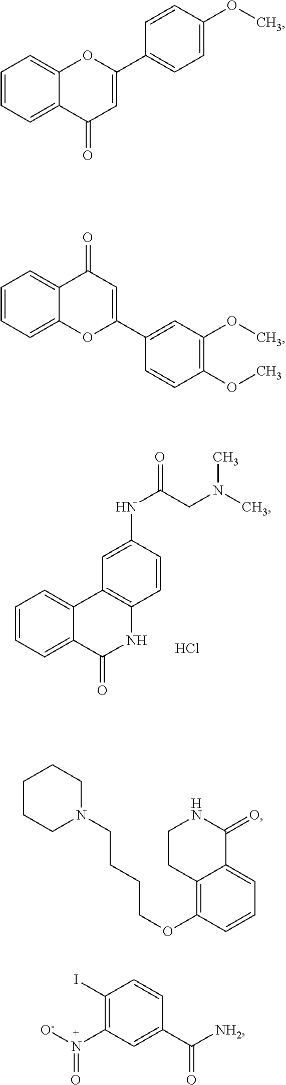

and a pharmaceutically acceptable salt thereof.

In another embodiment of the method, the PARP inhibitor is olaparib or a pharmaceutically acceptable salt thereof. In another embodiment, the PARP inhibitor is talazoparib or a pharmaceutically acceptable salt thereof.

In another embodiment of the method, the anti-CD123 antibody-drug conjugate is selected from the group consisting of an anti-CD123 antibody linked to a indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

In another embodiment of the method, the anti-CD123 anti-drug conjugate is the anti-CD123 antibody linked to indolinobenzodiazepine pseudodimer IMGN632.

In another embodiment of the method, the cancer is a hematological cancer. In an embodiment, the hematological cancer is selected from the group consisting of leukemia, lymphoma, and myeloma.

In another embodiment of the method, the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, Hodgkin's leukemia (HL), and myeloproliferative neoplasm (MPN). In an embodiment, the hematological cancer is acute myeloid leukemia (AML). In another embodiment, the acute myeloid leukemia is refractory acute myeloid leukemia (AML). In another embodiment, the acute myeloid leukemia is relapse acute myeloid leukemia.

In an embodiment of the method, the subject is human.

In an embodiment of the method, the anti-CD123 antibody drug conjugate and PARP inhibitor are administered to the subject simultaneously.

In an embodiment of the method, the anti-CD123 antibody drug conjugate and PARP inhibitor are administered to the subject sequentially.

A method for inducing cancer cell death is provided, the method comprising:

administering, to a population of cancer cells, an anti-CD123 antibody-drug conjugate and a poly ADP ribose (PARP) inhibitor in an amount effective to induce cancer cell death.

In an embodiment of the method, the PARP inhibitor is a PARP-1 inhibitor. In another embodiment, the PARP inhibitor is a PARP-2 inhibitor.

In another embodiment of the method, the PARP inhibitor is selected from the group consisting of:

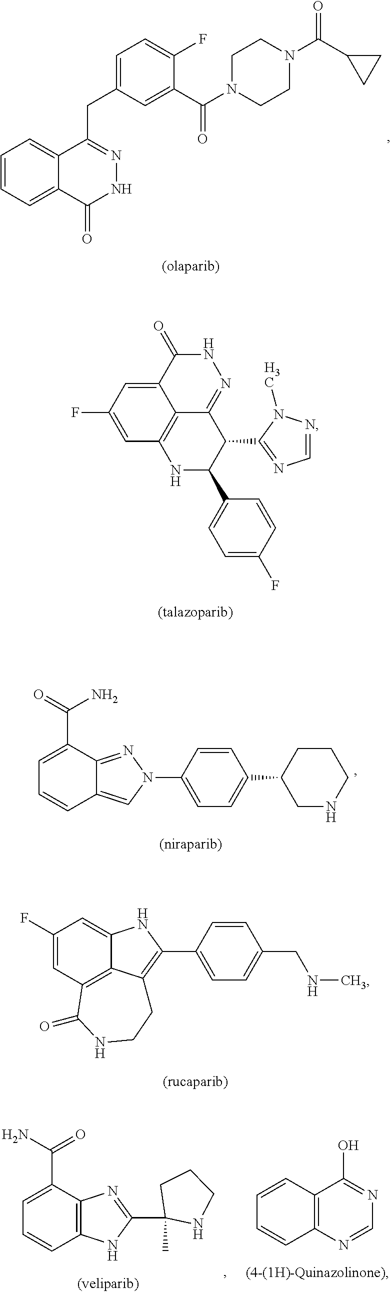

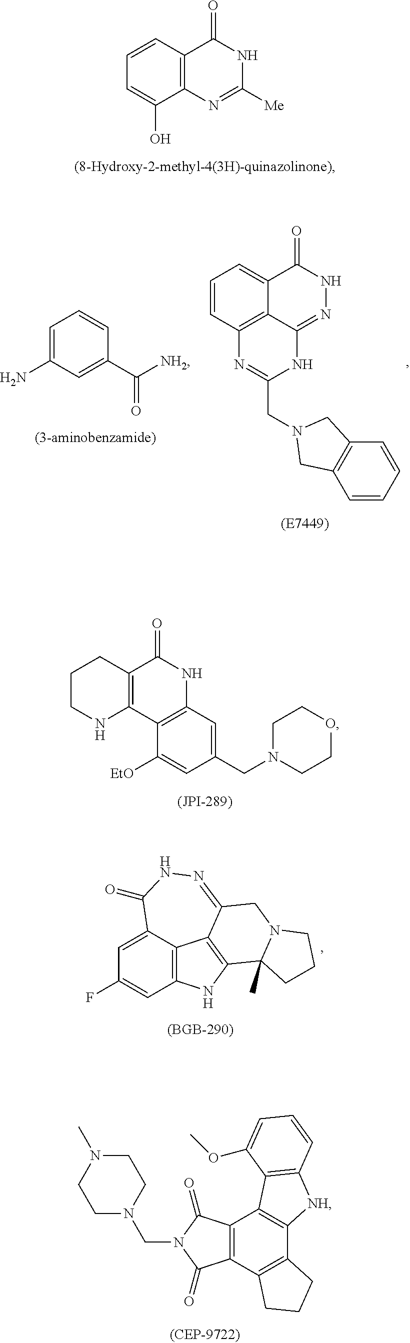 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

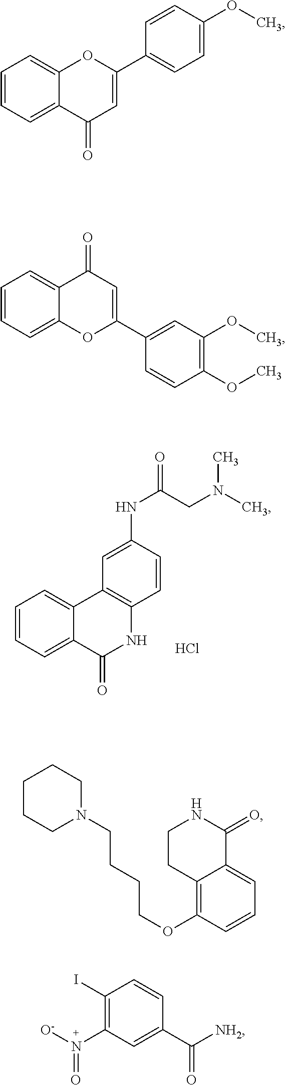

and pharmaceutically acceptable salts thereof.

In an embodiment of the method, the PARP inhibitor is olaparib or a pharmaceutically acceptable salt thereof. In another embodiment of the method, the PARP inhibitor is talazoparib or a pharmaceutically acceptable salt thereof.

In another embodiment of the method, the anti-CD123 antibody-drug conjugate is selected from the group consisting of an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

In another embodiment of the method, the anti-CD123 anti-drug conjugate is the anti-CD123 antibody linked to indolinobenzodiazepine pseudodimer IMGN632.

In another embodiment of the method, the cancer cells are hematological cancer cells. In another embodiment, the hematological cancer is selected from the group consisting of leukemia, lymphoma, and myeloma. In another embodiment, the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, Hodgkin's leukemia (HL), and myeloproliferative neoplasm (MPN).

In another embodiment of the method, the hematological cancer cells are acute myeloid leukemia cells. In another embodiment, the acute myeloid leukemia cells are refractory acute myeloid leukemia cells. In another embodiment, the acute myeloid leukemia cells are relapse acute myeloid leukemia cells.

In an embodiment of the method, the administering is carried out in vivo.

A composition is also provided comprising:

an anti-CD123 antibody-drug conjugate; and

a poly ADP ribose (PARP) inhibitor,

for use in therapy and/or as a medicament.

Also provided are an anti-CD123 antibody-drug conjugate and a poly ADP ribose (PARP) inhibitor for use in treating cancer.

Also provided are an anti-CD123 antibody-drug conjugate and a poly ADP ribose (PARP) inhibitor for use in inducing cancer cell death in a population of cancer cells.

### 4. BRIEF DESCRIPTION OF THE DRAWINGS

Embodiments are described herein with reference to the accompanying drawings, in which similar reference characters denote similar elements throughout the several views. It is to be understood that in some instances, various aspects of the embodiments may be shown exaggerated, enlarged, exploded, or incomplete to facilitate an understanding of the invention.

FIG. 1 shows a depiction of IMGN632 antibody conjugate.

FIG. 2 shows, on the left, results from an experiment described in Example 6.1. The table on the right shows the P value for the results. These results demonstrate that combination therapy using PARP inhibitors resulted in enhanced anti-leukemic effects over monotherapy. IMGN632+talazoparib significantly induced apoptosis in human acute myelogenous leukemia (AML) cell lines. See Example 6.1 for details.

### 5. DETAILED DESCRIPTION

A drug therapy combination is provided that comprises an anti-CD123 antibody-drug conjugate and a poly ADP ribose (PARP) inhibitor. In one embodiment, the drug therapy combination is formulated together in a single pharmaceutical composition. In another embodiment, the components of the drug therapy combination are formulated as separate pharmaceutical compositions.

An aspect of the invention is directed towards a composition comprising: an anti-CD123 antibody-drug conjugate, and a poly ADP ribose (PARP) inhibitor for use in therapy and/or as a medicament.

Another aspect of the invention is directed towards an anti-CD123 antibody-drug conjugate and a poly ADP ribose (PARP) inhibitor for use in treating cancer.

Another aspect of the invention is directed towards an anti-CD123 antibody-drug conjugate and a poly ADP ribose (PARP) inhibitor for use in inducing cancer cell death in a population of cancer cells.

Preferably, the PARP inhibitor is a PARP-1 inhibitor.

Preferably, the PARP inhibitor is a PARP-2 inhibitor.

Preferably, the PARP inhibitor is selected from the group consisting of:

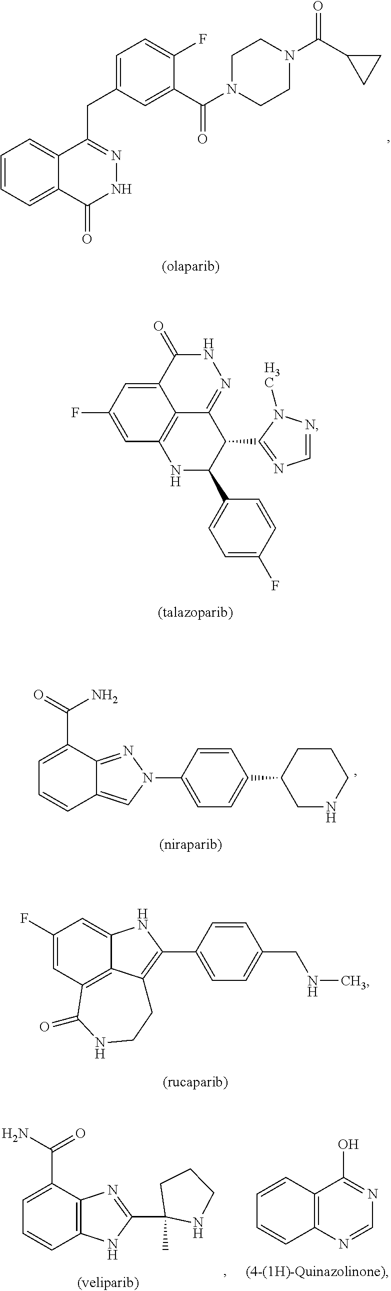

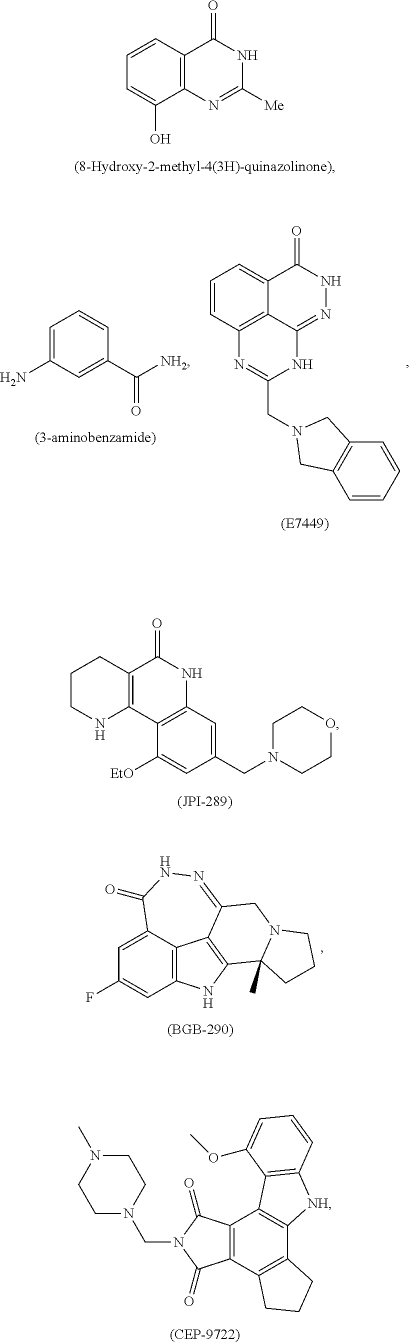 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

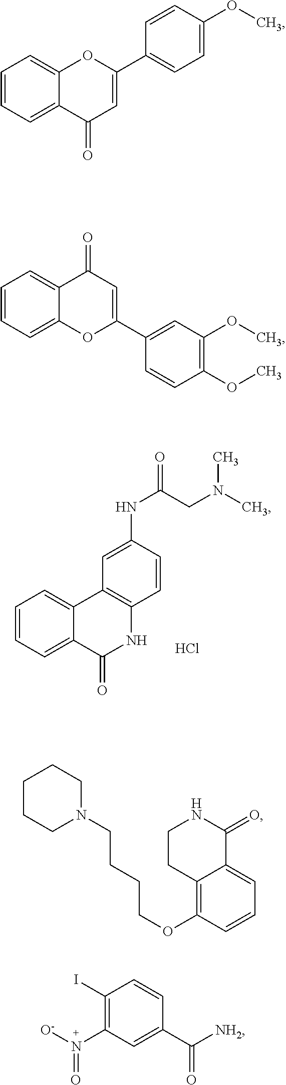

and a pharmaceutically acceptable salt thereof.

Preferably, the PARP inhibitor is olaparib or a pharmaceutically acceptable salt thereof.

Preferably, the PARP inhibitor is talazoparib or a pharmaceutically acceptable salt thereof.

Preferably, the anti-CD123 antibody-drug conjugate is selected from an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

Preferably, the anti-CD123 anti-drug conjugate is the anti-CD123 antibody linked to the indolinobenzodiazepine pseudodimer IMGN632.

Preferably, the cancer is a hematological cancer or the cancer cells are hematological cancer cells.

Preferably, the cancer is selected from the group consisting of leukemia, lymphoma, and myeloma.

Preferably, the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL).

Preferably, the cancer is acute myeloid leukemia or the hematological cancer cells are acute myeloid leukemia cells.

Preferably, the acute myeloid leukemia is refractory acute myeloid leukemia or the acute myeloid leukemia cells are refractory acute myeloid leukemia cells.

Preferably, the acute myeloid leukemia is relapse acute myeloid leukemia or the acute myeloid leukemia cells are relapse acute myeloid leukemia cells.

Preferably, the subject is human.

Preferably, said anti-CD123 antibody drug conjugate and PARP inhibitor are administered to said subject simultaneously.

Preferably, said anti-CD123 antibody drug conjugate and PARP inhibitor are administered to said subject sequentially.

Preferably, the cancer cells are hematological cancer cells.

Preferably, said administering is carried out in vivo.

Anti-CD123 Antibodies, Antibody Derivatives and Fragments, and Epitope-Binding Fragments

A pharmaceutical composition is provided comprising an anti-CD123 antibody-drug conjugate (ADC); and a poly ADP ribose (PARP) inhibitor.

As used herein, an anti-CD123 antibody-drug conjugate (ADC) comprises an anti-CD123 antibody (also referred to herein as “anti-CD123 antibody”) or antigen binding fragment thereof covalently linked via a stable linker molecule to a cytotoxic payload. CD123, also known as Interleukin-3 receptor subunit alpha, is a cell membrane protein that is a therapeutic target for a number of cancers, in particular hematological cancers, due to its high level of expression on cancerous, but not healthy cells. The amino acid sequence of human CD123 is provided below as SEQ ID NO: 1 (UniProtKB Accession No. P26951).

(SEQ ID NO: 1)
MVLLWLTLLLIALPCLLQTKEDPNPPITNLRMKAKAQQLTWDLNRNVTD
IECVKDADYSMPAVNNSYCQFGAISLCEVTNYTVRVANPPFSTWILFPE
NSGKPWAGAENLTCWIHDVDFLSCSWAVGPGAPADVQYDLYLNVANRRQ
QYECLHYKTDAQGTRIGCRFDDISRLSSGSQSSHILVRGRSAAFGIPCT
DKFVVFSQIEILTPPNMTAKCNKTHSFMHWKMRSHFNRKFRYELQIQKR
MQPVITEQVRDRTSFQLLNPGTYTVQIRARERVYEFLSAWSTPQRFECD
QEEGANTRAWRTSLLIALGTLLALVCVFVICRRYLVMQRLFPRIPHMKD
PIGDSFQNDKLVVWEAGKAGLEECLVTEVQVVQKT

The extracellular domain of CD123 comprises amino acid residues 19-305 of SEQ ID NO: 1. In one embodiment, the anti-CD123 antibody portion of the anti-CD123 conjugate binds to an epitope of CD123 located in residues 19-305.

The anti-CD123 antibody of the anti-CD123 ADC can be any full-length anti-CD123 antibody, an epitope binding fragment of a full-length anti-CD123 antibody, or an anti-CD123 antibody derivative. Preferably the anti-CD123 antibody is a monoclonal antibody.

Full-length or intact anti-CD123 monoclonal antibodies comprise a tetramer which is usually composed of at least two heavy (H) chains and at least two light (L) chains. Each heavy chain is comprised of a heavy chain variable (V_{H}) region and a heavy chain constant (C_{H}) region, usually comprised of three domains (C_{H}1, C_{H}2 and C_{H}3 domains). Heavy chains can be of any isotype, including IgG (IgG1, IgG2, IgG3 and IgG4 subtypes), IgA (IgA1 and IgA2 subtypes), IgM and IgE. In one embodiment, the anti-CD123 antibody portion of the anti-CD123 ADC is an IgG antibody. Each light chain of the antibody is comprised of a light chain variable (V_{L}) region and a light chain constant (C_{L}) region. Light chains include kappa chains and lambda chains. The heavy and light chain variable regions are responsible for CD123 antigen recognition, while the heavy and light chain constant regions may mediate the binding of the immunoglobulin to host tissues or factors, including various cells of the immune system (e.g., effector cells) and the first component (C1q) of the classical complement system.

The V_{H} and V_{L} regions are further subdivided into regions of hypervariability, termed “complementarity determining regions,” or “CDRs,” that are interspersed with regions of more conserved sequence, termed “framework regions” (FR). Each V_{H} and V_{L} region is composed of three CDR domains and four FR domains arranged from amino-terminus to carboxy-terminus in the following order: FR1-CDR1-FR2-CDR2-FR3-CDR3-FR4. The variable regions of the heavy and light chains contain a binding domain that interacts with CD123.

Suitable anti-CD123 antibody fragments (including Fab and (Fab)₂ fragments) that exhibit epitope-binding ability can be obtained using methods known in the art, for example, by protease cleavage of intact antibodies. Single domain antibody fragments possess only one variable domain (e.g., V_{L} or V_{H}). Examples of the epitope-binding fragments suitable for inclusion in the anti-CD123 ADC as described herein include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; and (iv) Fv fragments consisting essentially of a V_{L} and V_{H} domain.

Additional examples of epitope-binding fragments suitable for inclusion in the anti-CD123 ADC as described herein are (v) dAb fragments (see, e.g., Ward et al. “Binding Activities Of A Repertoire Of Single Immunoglobulin Variable Domains Secreted From Escherichia coli,” Nature 341:544-546 (1989)), which consist essentially of a V_{H} or V_{L} domain and are also called domain antibodies (see, e.g., Holt et al. “Domain Antibodies: Proteins For Therapy,” Trends Biotechnol. 21(11):484-490 (2003).

Additional examples of epitope-binding fragments suitable for inclusion in the anti-CD123 ADC as described herein are (vi) camelid or nanobodies (see, e.g., Revets et al. “Nanobodies As Novel Agents For Cancer Therapy,” Expert Opin. Biol. Ther. 5(1):111-124 (2005)) and (vii) isolated complementarity determining regions (CDR).

An epitope-binding fragment may contain 1, 2, 3, 4, 5 or all 6 of the CDR domains of such antibody.

Such antibody fragments are obtained using conventional techniques known to those of skill in the art. For example, F(ab′)2 fragments may be generated by treating an antibody with pepsin. The resulting F(ab′)2 fragment may be treated to reduce disulfide bridges to produce Fab′ fragments. Fab fragments may be obtained by treating an IgG antibody with papain and Fab′ fragments may be obtained with pepsin digestion of IgG antibody. A Fab′ fragment may be obtained by treating an F(ab′)₂ fragment with a reducing agent, such as dithiothreitol. Antibody fragments may also be generated by expression of nucleic acids encoding such fragments in recombinant cells, which is a method known in the art (see e.g., Evans et al. “Rapid Expression Of An Anti-Human C5 Chimeric Fab Utilizing A Vector That Replicates In COS And 293 Cells,” J. Immunol. Meth. 184:123-38 (1995)). For example, a chimeric gene encoding a portion of an F(ab′)₂ fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Suitable anti-CD123 antibody derivatives include those molecules that contain at least one epitope-binding domain of an antibody, and are typically formed using recombinant techniques. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

One exemplary antibody derivative includes a single chain Fv (scFv). A scFv is formed, using methods known in the art, from the two domains of the Fv fragment, the V_{L} region and the V_{H} region, which are encoded by separate genes. Such gene sequences or their encoding cDNA are joined, using recombinant methods known in the art, by a flexible linker (typically of about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988)). Alternatively, by employing a flexible linker that is too short (e.g., less than about 9 residues) to enable the V_{L} and V_{H} regions of a single polypeptide chain to associate together, one can form a bispecific antibody.

In another embodiment, the anti-CD123 antibody derivative is a divalent or bivalent single-chain variable fragment, engineered using methods known in the art, by linking two scFvs together either in tandem (i.e., tandem scFv), or such that they dimerize to form diabodies (Holliger et al. “‘Diabodies’: Small Bivalent And Bispecific Antibody Fragments,” Proc. Natl. Acad. Sci. (U.S.A.) 90(14), 6444-8 (1993)). In yet another embodiment, the anti-CD123 antibody is a trivalent single chain variable fragment, and is engineered using methods known in the art by linking three scFvs together, either in tandem or in a trimer formation to form triabodies. In another embodiment, the antibody is a tetrabody single chain variable fragment. In another embodiment, the antibody is a “linear antibody” which is an antibody comprising a pair of tandem Fd segments (V_{H}—C_{H}1-V_{H}-C_{H}1) that form a pair of antigen binding regions (see Zapata et al. Protein Eng. 8(10):1057-1062 (1995)). In another embodiment, the antibody derivative is a minibody, consisting of the single-chain Fv regions coupled to the C_{H}3 region (i.e., scFv-C_{H}3). Methods for engineering minibodies are known in the art.

In one embodiment, anti-CD123 antibody or antigen binding fragment thereof is a human antibody. In another embodiment, the anti-CD123 antibody or antigen binding fragment thereof is a “humanized” antibody. The term “humanized” refers to a chimeric molecule, generally prepared using recombinant techniques known in the art, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified, using methods known in the art, to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A. F. et al. “Mouse/Human Chimeric Monoclonal Antibody In Man: Kinetics And Immune Response,” Proc. Natl. Acad. Sci. USA 86:4220-4224 (1989)).

Another approach focuses not only on providing human-derived constant regions, but modifying the variable regions so as to reshape them as closely as possible to human form. The CDRs are flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody on the FRs present in the human antibody to be modified. Application of this approach to various antibodies has been reported by Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M. S. et al., “Chimeric And Humanized Antibodies With Specificity For The CD33 Antigen,” J. Immunol. 148:1149-1154 (1992). The ability to humanize an antigen is well-known (see, e.g., U.S. Pat. Nos. 5,225,539; 5,530,101; 5,585,089; 5,859,205; 6,407,213; 6,881,557).

Exemplary anti-CD123 antibodies, epitope binding fragments thereof, and antibody derivatives known in the art and suitable for inclusion in ADC of the therapeutic drug combination as described herein include, but are not limited to, anti-CD123 antibodies described by Han et al., “Antileukemia Efficacy and Mechanisms of Action of SL-101, a Novel Anti-CD123 Antibody Conjugate, in Acute Myeloid Leukemia,” Clin Cancer Res 23(13):3385-3395 (2017); Oon et al., “A cytotoxic anti-IL-3Rα antibody targets key cells and cytokines implicated in systemic lupus erythematosus,” JCI Insight 1(6):e86131 (2016); Moradi-Kalbolandi et al., “Soluble Expression and Characterization of a New scFv Directed to Human CD123,” Appl Biochem Biotechnol 178(7):1390-406 (2016); He et al., “A Phase 1 study of the safety, pharmacokinetics and anti-leukemic activity of the anti-CD123 monoclonal antibody CSL360 in relapsed, refractory or high-risk acute myeloid leukemia,” Leuk Lymphoma 56(5):1406-15 (2015); Kuo et al., “Antibody internalization after cell surface antigen binding is critical for immunotoxin development,” Bioconjug Chem 20(10):1975-82 (2009); Du et al., “New immunotoxins targeting CD123, a stem cell antigen on acute myeloid leukemia cells,” J Immunother. 30(6):607-13 (2007).

Cytotoxic Payload Portion of the Anti-CD123 Antibody-Drug Conjugate

The cytotoxic payload portion of the anti-CD123 ADC of the pharmaceutical compositions and methods disclosed herein can be any small molecule drug that is stable, highly toxic, and capable of inducing target cell death after being internalized by the target cell and released. Suitable payload molecules include, but are not limited to, biologically active anti-microtubule agents, alkylating agents, and DNA minor groove binding agents. Exemplary payload molecules include, but are not limited to, maytanisinoids (tubulin inhibitor), auristatins and auristatin analogues (tubulin inhibitor), dolastatins (tubulin inhibitor), cryptophycin (tubulin inhibitor), enediyne antibiotics, e.g., esperamicin and calicheamicin (induce DNA double strand breaks), pyrolobenodiazepine (PBD) (DNA minor groove binding agent), duocarmycin (DNA minor groove binding agent), indolinobenzodiazepines (alkylating agent), doxorubicin, SN38 (topoisomerase I inhibitor), and DXd (exatecan derivative topoisomerase inhibitor).

The portion of the ADC linking the anti-CD123 antibody to its cytotoxic payload is a stable linker. The linker may be cleavable or non-cleavable, pH sensitive or non-pH sensitive. Suitable linkers include, disulfide linkers, non-cleavable thioether linkers, peptide linkers, β-glucuronide linkers, etc. Exemplary linkers known in the art and suitable for use in the anti-CD123 ADC as described herein include, but are not limited to, Fmoc-Val-Cit-PAB, Fmoc-Val-Cit-PAB-PNP, Mc-Val-Cit-PABC-PNP, Val-Cit-PAB, Mc-Val-Cit-PAB-PNP, SMCC, (Ac)Phe-Lys(Alloc)-PABC-PNP, 6-Maleimidohexanoic acid N-hydroxysuccinimide ester(ECMS), Phe-Lys(Trt)-PAB, Fmoc-Phe-Lys(Trt)-PAB, Ala-Ala-Asn-PAB TFA salt, Fmoc-Ala-Ala-Asn-PAB-PNP, Fmoc-Gly3-Val-Cit-PAB, Py-ds-Prp-Osu, Py-ds-dmBut-Osu, MAL-HA-Osu, MEL-tri-EG-OPFP, MEL-di-EG-OPFP, BCOT-di-EG-OPFP, BCOT-tri-EG-OPFP, BCOT-tetra-EG-OPFP, N3-di-EG-OPFP, N3-tri-EG-OPFP, N3-tetra-EG-OPFP, ALD-BZ-Osu, ALD-di-EG-Osu, ALD-tetra-EG-Osu, ALD-di-EG-OPFP, ALD-tetra-EG-OPFP, PHA-di-EG-OPFP, PHA-tetra-EG-OPFP.

The synergistic anti-tumor effects of the anti-CD123 ADC are dependent on the DNA damaging component of the antibody-drug conjugate and not on the antibody specificity. Only alterations in the sequence identity that impact on the ability of the anti-CD123 antibody to bind to leukemia cells would affect efficacy of the antibody-drug conjugate. Such alterations in sequence identity and the ability of an antibody to bind to cancer and/or tumor cells of interest (e.g., leukemia cells) can be tested by the skilled artisan using methods known in the art. Further, heavy chains of different isotypes (in addition to IgG) can be used in anti-CD123 ADCs. As long as the antibody comprising the heavy chain binds to cancer and/or tumor cells of interest, it can be efficacious for use in an anti-CD123 ADC.

Anti-CD123 Antibody-Drug Conjugate (ADC) Linked to an Indolinobenzodiazepine Pseudodimer Payload

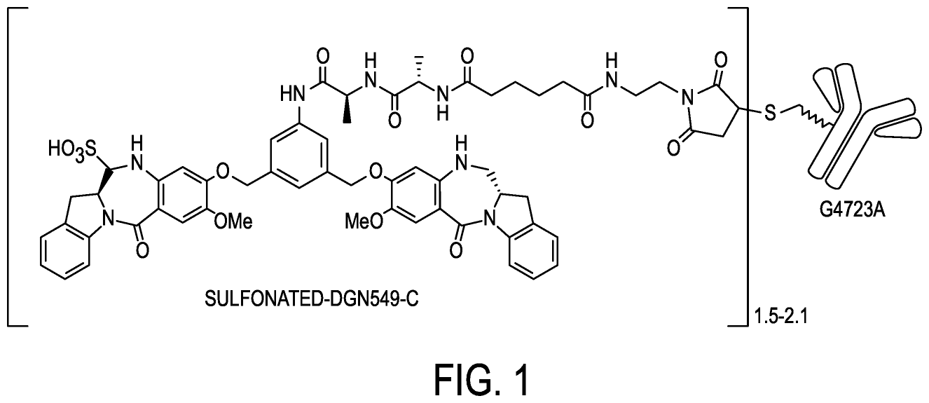

In one embodiment of the pharmaceutical compositions and methods disclosed herein, the anti-CD123 antibody-drug conjugate (ADC) of the therapeutic drug combination comprises an anti-CD123 antibody linked to an indolinobenzodiazepine pseudodimer payload. An exemplary anti-CD123 ADC of this form is known in the art as IMGN632 (ImmunoGen, Inc.). See U.S. Pat. No. 10,077,313 to Kovtun et al.; Kovtun et al., “A CD123-targeting antibody-drug conjugate, IMGN632, designed to eradicate AML while sparing normal bone marrow cells,” Blood Adv. 2(8): 848-858 (2018); Miller et al, “A DNA-Interacting Payload Designed to Eliminate Cross-Linking Improves the Therapeutic Index of Antibody—Drug Conjugates (ADCs),” Molecular Cancer Therapeutics 17(3): 650-660 (2018), and US20160095938A1 to Fishkin. A depiction of IMGN632 antibody conjugate is shown in FIG. 1.

The anti-CD123 monoclonal antibody of the IMGN632 ADC has a V_{H} domain comprising a heavy chain complementary determining region-1 (H-CDR1) having the amino acid sequence of SSIMH (SEQ ID NO: 2), or a modified amino acid sequence of SEQ ID NO: 2, said modified sequence containing 1 or 2 amino acid residue modifications as compared SEQ ID NO: 2; a H-CDR2 having the amino acid sequence of YIKPYNDGTKYNEKFKG (SEQ ID NO: 3), or a modified amino acid sequence of SEQ ID NO: 3, said modified sequence containing 1, 2, or 3 amino acid residue modifications as compared to SEQ ID NO: 3; and a H-CDR3 having the amino acid sequence of EGGNDYYDTMDY (SEQ ID NO: 4), or a modified amino acid sequence of SEQ ID NO: 4, said modified sequence containing 1 or 2 amino acid residue modifications as compared to SEQ ID NO: 4.

In one embodiment, the IMGN632 antibody ADC comprises an anti-CD123 monoclonal antibody having a variable heavy chain region comprising the amino acid sequence of SEQ ID NO:8 as shown below with the CDRs underlined:

(SEQ ID NO: 8)
QVQLVQSGAEVKKPGASVKVSCKASGYIFTSSIMHWVRQAPGQGLEWIG
YIKPYNDGTKYNEKFKGRATLTSDRSTSTAYMELSSLRSEDTAVYYCAR
EGGNDYYDTMDYWGQGTLVTVSS

The anti-CD123 monoclonal antibody of the IMGN632 ADC comprises a V_{L} region, where the V_{L} region comprises a light chain complementary determining region 1 (L-CDR1) having the amino acid sequence of RASQDINSYLS (SEQ ID NO: 5), or a modified amino acid sequence of SEQ ID NO: 5, said modified sequence containing 1, 2, or 3 amino acid residue modifications as compared to SEQ ID NO: 5; a L-CDR2 having the amino acid sequence of RVNRLVD (SEQ ID NO: 6), or a modified amino acid sequence of SEQ ID NO: 6, said modified sequence containing 1 or 2 amino acid residue modifications as compared to SEQ ID NO: 6; and a L-CDR3 having the amino acid sequence of LQYDAFPYT (SEQ ID NO: 7), or a modified amino acid sequence of SEQ ID NO: 7, said modified sequence containing 1 or 2 amino acid residue modifications as compared to SEQ ID NO: 7.

In one embodiment, the IMGN632 antibody ADC comprises an anti-CD123 monoclonal antibody having a variable light chain region comprising the amino acid sequence of SEQ ID NO: 9 as shown below with the CDRs underlined:

(SEQ ID NO: 9)
DIQMTQSPSSLSASVGDRVTITCRASQDINSYLSWFQQKPGKAPKTLIY
RVNRLVDGVPSRFSGSGSGNDYTLTISSLQPEDFATYYCLQYDAFPYTF
GQGTKVEIKR

In another embodiment, the anti-CD123 monoclonal antibody of the IMGN632 ADC comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity with SEQ ID NO: 8, and/or a light chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity with SEQ ID NO: 9.

In one embodiment, the anti-CD123 monoclonal antibody of the IMGN632 ADC is the G4723A antibody comprising a full length heavy chain of SEQ ID NO: 10 and a full length light chain of SEQ ID NO: 11 as provided below. See U.S. Pat. No. 10,077,313 to Kovtun et al. The CDR regions within each full length sequence are underlined.

(SEQ ID NO: 10)
QVQLVQSGAEVKKPGASVKVSCKASGYIFTSSIMHWVRQAPGQGLEWIG
YIKPYNDGTKYNEKFKGRATLTSDRSTSTAYMELSSLRSEDTAVYYCAR
EGGNDYYDTMDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLCLSPG
(SEQ ID NO: 11)
DIQMTQSPSSLSASVGDRVTITCRASQDINSYLSWFQQKPGKAPKTLIY
RVNRLVDGVPSRFSGSGSGNDYTLTISSLQPEDFATYYCLQYDAFPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

As shown in FIG. 1, the sulfur adjacent to the G4723A antibody is derived from site specific engineered cysteine residues on the heavy chain constant region of the antibody.

Anti-CD123 Antibody-Drug Conjugate (ADC) Linked to a Pyrrolobenzodiazepine Dimer Payload

Another suitable anti-CD123 ADC suitable for inclusion in the therapeutic drug combination of the pharmaceutical compositions and methods disclosed herein includes an anti-CD123 antibody linked to a pyrrolobenzodiazepine dimer payload. An exemplary anti-CD123 ADC of this form is known in the art as SGN-CD123A (Seattle Genetics, Inc.). See Li et al., “Characterization of SGN-CD123A, a potent CD123-directed antibody-drug conjugate for acute myeloid leukemia,” Mol Cancer Ther. 17(2):554-564 (2018); Sutherland et al., “SGN-CD123A, a Pyrrolobenzodiazepine Dimer Linked Anti-CD123 Antibody Drug Conjugate, Demonstrates Effective Anti-Leukemic Activity in Multiple Preclinical Models of AML,” Blood 126(23):330 (2015); and US20180169261A1 to Sutherland.

Anti-CD123 Antibody-Drug Conjugate (ADC) Linked to a Camptothecin Payload

Other suitable anti-CD123 ADCs suitable for inclusion in the therapeutic drug combination include an anti-CD123 antibody linked to camptothecin as described by Li et al., Design, synthesis and evaluation of anti-CD123 antibody drug conjugates,” Bioorg Med Chem 24:5855-60 (2016); an anti-CD123 antibody linked to a duorcarmycin derivative (Synthon Biopharmaceuticals B.V.) as described in WO2015177360A1 to Ariaans; and an anti-CD123 antibody linked to cyclopropylpyrroloindoline as described by Ha et al., “Generation and preclinical characterization of CD123-CPI antibody-drug conjugate (ADC),” Proceedings of the AACR Annual Meeting 78(13 suppl): 935 (2018).

Poly-ADP Ribose Polymerase (PARP) Inhibitors

The second component of the pharmaceutical composition is a PARP inhibitor. PARP refers to a family of poly-ADP ribose polymerases that participate in a variety of DNA related functions including cell proliferation, differentiation, apoptosis, DNA repair, and also has effects on telomere length and chromosome stability (d'Adda di Fagagna et al, Nature Gen., 23(1): 76-80 (1999)). A PARP inhibitor is a substance or substances that selectively bind to the poly-ADP ribose polymerase (PARP) enzyme and decrease its activity. In one aspect, the PARP inhibitor of the composition and methods disclosed herein inhibits PARP-1 (i.e., a “PARP-1 inhibitor”). In another embodiment, the PARP inhibitor of the composition and methods disclosed herein inhibits PARP-2 (i.e., a “PARP-2 inhibitor”).

Suitable PARP inhibitors include those which are designed as analogs of benzamides, which bind competitively with the natural substrate NAD in the catalytic site of PARP. These PARP inhibitors include, but are not limited to, benzamides, quinolones and isoquinolones, benzopyrones, methyl 3,5-diiodo-4-(4′-methoxy-3′,5′-diiodo-phenoxy) benzoate (see, e.g., U.S. Pat. Nos. 5,464,871, 5,670,518, 5,922,775, 6,017,958, 5,736,576, and 5,484,951). Other suitable PARP inhibitors include a variety of cyclic benzamide analogs (i.e. lactams) which are potent inhibitors at the NAD site.

Other PARP inhibitors include, but are not limited to, benzimidazoles and indoles (see, e.g., EP 841924, EP 127052, U.S. Pat. Nos. 6,100,283, 6,310,082, US 2002/156050, US2005/054631, WO 05/012305, WO 99/11628, and US 2002/028815).

Exemplary PARP inhibitors include, but are not limited to, isoquinolinone and dihydrolisoquinolinone as disclosed in U.S. Pat. No. 6,664,269 and WO 99/11624. Other exemplary PARP inhibitors include, but are not limited to, nicotinamide, 3-aminobenzamide, monoaryl amides and bi-, tri-, or tetracyclic lactams, phenanthridinones (Perkins et al, Cancer Res., 61:4175-4183 (2001)), 3,4-dihydro-5-methyl-isoquinolin-1(2H)-one and benzoxazole-4-carboxamide (Griffin et al., Anticancer Drug Des, 10:507-514 (1995); Griffin et al, J Med Chem, 41:5247-5256 (1998); and Griffin et al, Pharm Sci, 2:43-48 (1996)), dihydroisoquinolin-1(2H)-nones,1,6-naphthyridine-5(6H)-ones, quinazolin-4(3H)-ones, thieno[3,4-c]pyridin-4(5H)ones and thieno[3,4-d]pyrimidin-4(3H)-ones, 1,5-dihydroxyisoquinoline, and 2-methyl-quinazolin-4[3H]-one (Yoshida et al., J Antibiot (Tokyo) 44: 111-112 (1991); Watson et al, Bioorg Med Chem., 6:721-734 (1998); and White et al, J Med Chem., 43:4084-4097 (2000)), 1,8-Napthalimide (Banasik et al, J Biol Chem., 267: 1569-1575 (1992); Li et al, Bioorg Med Chem Lett, 11:1687-1690 (2001)), tetracyclic lactams, 1,11b-dihydro-[1]benzopyrano-[4,3,2-de]isoquinolin-3[2H]-one, l-methyl-4-phenyl-1,2,3,6-tetrahydropyridine (MPTP) (Zhang et al., Biochem Biophys Res Commun., 278:590-598 (2000); and Mazzon et al, Eur J Pharmacol, 415:85-94 (2001)). Other examples of PARP inhibitors include, but are not limited to, those detailed in the patents: U.S. Pat. Nos. 5,719,151, 5,756,510, 6,015,827, 6,100,283, 6,156,739, 6,310,082, 6,316,455, 6,121,278, 6,201,020, 6,235,748, 6,306,889, 6,346,536, 6,380,193, 6,387,902, 6,395,749, 6,426,415, 6,514,983, 6,723,733, 6,448,271, 6,495,541, 6,548,494, 6,500,823, 6,664,269, 6,677,333, 6,903,098, 6,924,284, 6,989,388, 6,277,990, 6,476,048, and 6,531,464. Additional examples of PARP inhibitors include, but are not limited to, those detailed in the patent application publications: US 2004198693 A1, US 2004034078A1, US 2004248879A1, US 2004249841A 2005080096A1, US 2005171101A1, US 2005054631A1, WO 05054201A1, WO 05054209A1, WO 05054210A1, WO 05058843A1, WO 06003146A1, WO 06003147A1, WO 06003148A1, WO 06003150A1, and WO 05097750A1.

Exemplary PARP inhibitors of the pharmaceutical composition and methods as described herein include, but are not limited to,

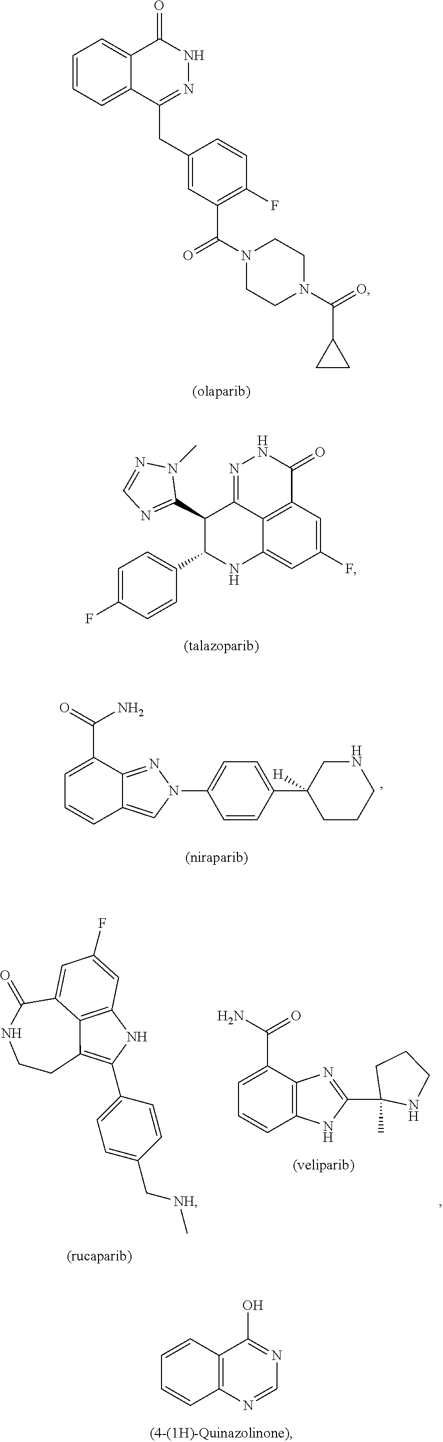 [SMILES: O=C(c1cc(Cc2n[nH]c(=O)c3ccccc23)ccc1F)N1CCN(C(=O)C2CC2)CC1 ; Cn1ncnc1[C@H]1c2n[nH]c(=O)c3cc(F)cc(c23)N[C@@H]1c1ccc(F)cc1 ; NC(=O)c1cccc2cn(-c3ccc([C@@H]4CCCNC4)cc3)nc12 ; CNCc1ccc(-c2[nH]c3cc(F)cc4c3c2CCNC4=O)cc1 ; C[C@]1(c2nc3c(C(N)=O)cccc3[nH]2)CCCN1 ; Oc1ncnc2ccccc12]

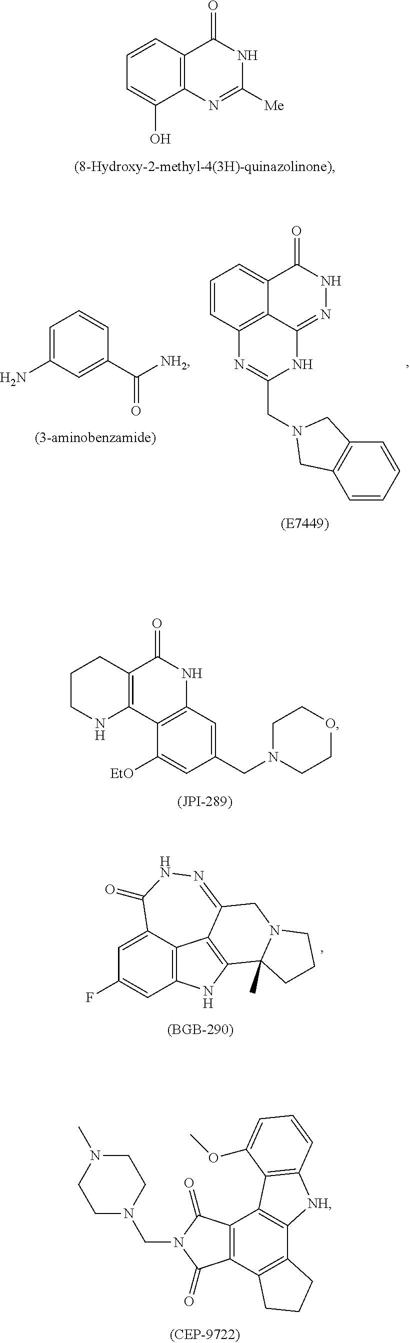 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

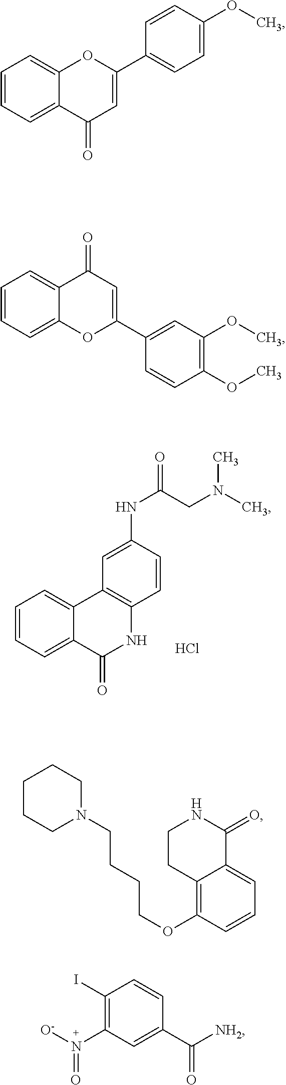

and
a pharmaceutically acceptable salt thereof.

In one embodiment, the PARP inhibitor of the composition and methods as described herein is olaparib. In another embodiment, the PARP inhibitor of the composition and methods as described herein is talazoparib. In another embodiment, the PARP inhibitor of the composition and methods as described herein is niraparib. In another embodiment, the PARP inhibitor of the composition and methods as described herein is rucaparib. In another embodiment, the PARP inhibitor of the composition and methods as described herein is veliparib.

A “pharmaceutically acceptable salt” as used herein, refers to a pharmaceutically acceptable organic or inorganic salt of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts.

A pharmaceutically acceptable salt may include or comprise another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. In particular embodiments, the pharmaceutically acceptable salt is a sodium salt or a potassium salt.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A method for treating cancer in a subject is also provided. In an embodiment, the method comprises administering, to the subject, a therapeutic drug combination comprising an anti-CD123 antibody-drug conjugate and poly ADP ribose (PARP) inhibitor as disclosed herein, in an amount effective to treat the cancer.

A method for inducing cancer cell death is also provided, the method comprising administering, to a population of cancer cells, an anti-CD123 antibody-drug conjugate and a poly ADP ribose (PARP) inhibitor in an amount effective to induce cancer cell death.

A composition is also provided, the composition comprising an anti-CD123 antibody-drug conjugate, and a poly ADP ribose (PARP) inhibitor, for use in therapy and/or as a medicament.

An anti-CD123 antibody-drug conjugate and a poly ADP ribose (PARP) inhibitor is also provided for use in treating cancer.

An anti-CD123 antibody-drug conjugate and a poly ADP ribose (PARP) inhibitor is also provided for use in inducing cancer cell death in a population of cancer cells.

In one embodiment, the anti-CD123 ADC and PARP inhibitor are administered simultaneously. In another embodiment, the anti-CD123 ADC and PARP inhibitor are administered sequentially. In one embodiment, the anti-CD123 ADC and PARP inhibitor are administered as separate pharmaceutical compositions. In another embodiment, the anti-CD123 ADC and PARP inhibitor are administered together as a single pharmaceutical composition. In one embodiment, the anti-CD123 ADC and PARP inhibitor are administered via the same route of administration. In another embodiment, the anti-CD123 ADC and PARP inhibitor are administered via different routes of administration. Suitable routes of administration include, but are not limited to, intraarticular (in the joints), intravenous, intramuscular, intratumoral, intradermal, intraperitoneal, subcutaneous, orally, topically, intrathecally, inhalationally, transdermally, rectally, and the like.

In one embodiment, the subject has a hematological cancer, such as, but not limited to a leukemia, lymphoma, and myeloma. Suitable hematological cancers that can be treated in accordance with the methods and composition disclosed herein include, but are not limited to, acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, Hodgkin's leukemia (HL), and myeloproliferative neoplasm (MPN).

In one embodiment, the method provided herein can be used to treat acute myeloid leukemia. The acute myeloid leukemia can be, for example, newly diagnosed AML, refractory AML or relapse AML.

The subject being treating is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As disclosed above, anti-CD123 antibody-drug conjugate and PARP inhibitor is administered in an amount “effective” to treat the cancer. An effective amount of the anti-CD123 antibody-drug conjugate and PARP inhibitor can be determined using methods known in the art. An effective amount is the amount of the anti-CD123 antibody-drug conjugate and PARP inhibitor that elicits the desired biological response in a subject. Such response includes alleviation of the symptoms of the cancer being treated, inhibition or a delay in the recurrence of symptom of the cancer, an increase in the longevity of the subject compared to the absence of the treatment, or inhibition or delay in the progression of symptom of the cancer. The effective amount of the ADC and PARP inhibitor to be administered to a subject will depend on the stage, category and status of the cancer and characteristics of the subject, such as general health, age, sex, body weight and drug tolerance. The effective amount of the ADC and PARP inhibitor to be administered will also depend on administration route and dosage form. Dosage amount and interval can be adjusted individually to provide plasma levels of the active compound that are sufficient to maintain desired therapeutic effects. The terms “treatment,” “treat,” and “treating” refer to reversing, alleviating, or inhibiting the progress of a cancer, or one or more symptoms thereof, as described herein.

As described herein, treatment of a subject having cancer with the combination of the anti-CD123 ADC and PARP inhibitor has a synergistic therapeutic effect. In other words, the therapeutic effect, e.g., DNA damage, tumor cell apoptosis, cell cycle arrest of cancer cells, etc., that is observed with the combination treatment is greater than the sum of effects observed by treatment with the anti-CD123 ADC and PARP inhibitor alone.

An aspect of the invention is directed towards a composition comprising: an anti-CD123 antibody-drug conjugate, and a poly ADP ribose (PARP) inhibitor for use in therapy and/or as a medicament.

Another aspect of the invention is directed towards a CD123 antibody-drug conjugate and a poly ADP ribose (PARP) inhibitor for use in treating cancer.

Another aspect of the invention is directed towards a CD123 antibody-drug conjugate and a poly ADP ribose (PARP) inhibitor for use in inducing cancer cell death in a population of cancer cells.
Preferably, the PARP inhibitor is a PARP-1 inhibitor.
Preferably, the PARP inhibitor is a PARP-2 inhibitor.

Preferably, the PARP inhibitor is selected from the group consisting of:

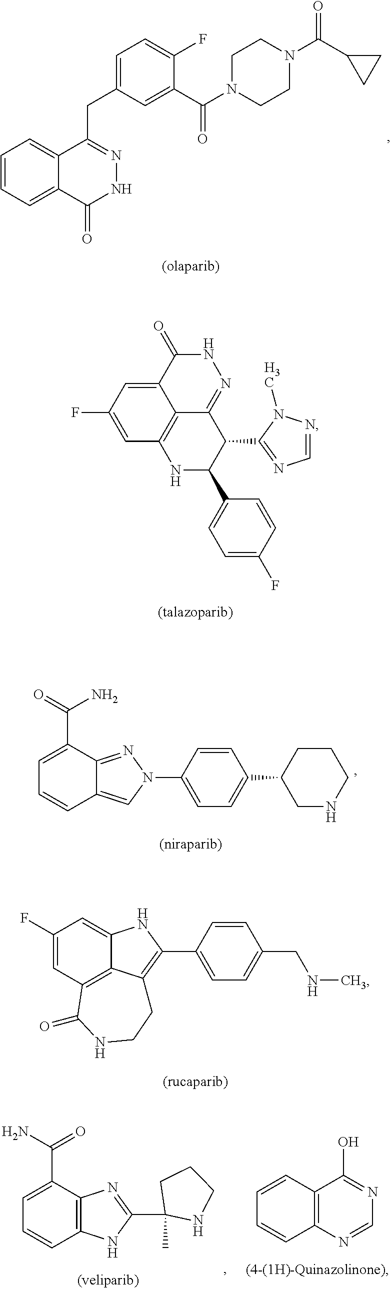

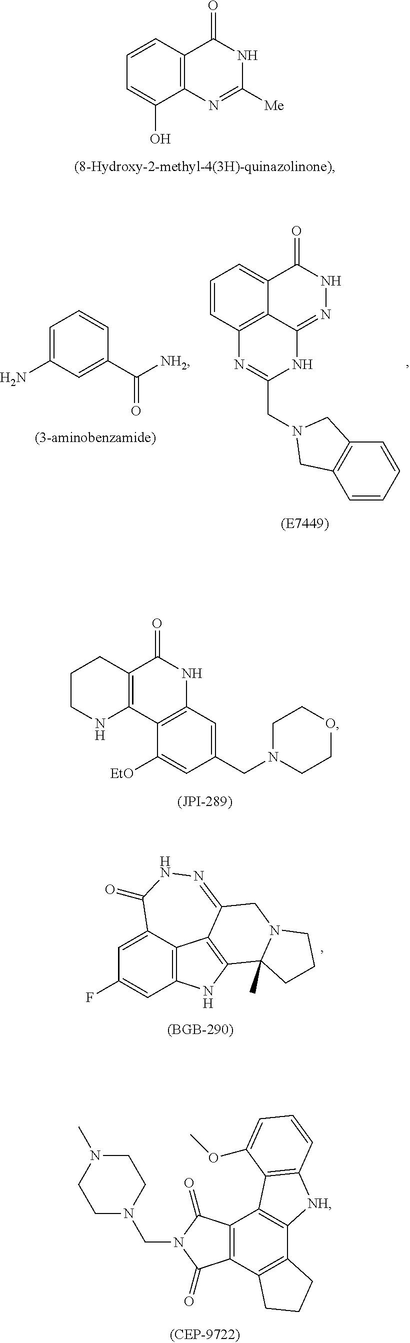 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4]

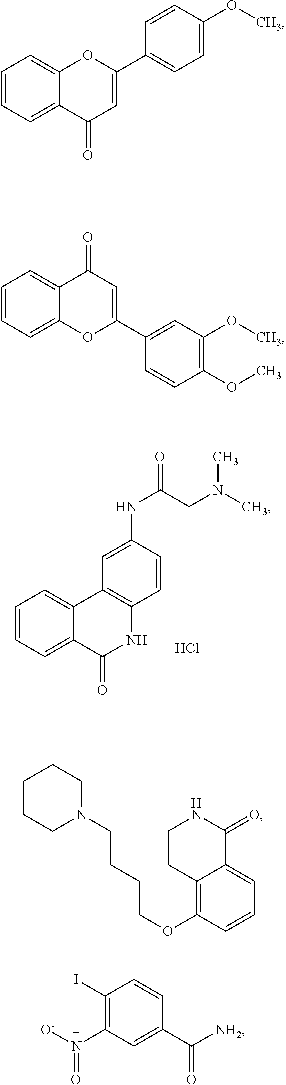

and pharmaceutically acceptable salts thereof.
Preferably, the PARP inhibitor is olaparib or a pharmaceutically acceptable salt thereof.
Preferably, the PARP inhibitor is talazoparib or a pharmaceutically acceptable salt thereof.

Preferably, the anti-CD123 antibody-drug conjugate is selected from an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

Preferably, the anti-CD123 anti-drug conjugate is the anti-CD123 antibody linked to indolinobenzodiazepine pseudodimer known as IMGN632.
Preferably, the cancer is a hematological cancer or the cancer cells are hematological cancer cells.
Preferably, the cancer is selected from the group consisting of leukemia, lymphoma, and myeloma.

Preferably, the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL).

Preferably, the cancer is or the acute myeloid leukemia or the hematological cancer cells are acute myeloid leukemia cells.

Preferably, the acute myeloid leukemia is refractory acute myeloid leukemia or the acute myeloid leukemia cells are refractory acute myeloid leukemia cells.

Preferably, the acute myeloid leukemia is relapse acute myeloid leukemia or the acute myeloid leukemia cells are relapse acute myeloid leukemia cells.

Preferably, the subject is human.

Preferably, said anti-CD123 antibody drug conjugate and PARP inhibitor are administered to said subject simultaneously.

Preferably, said anti-CD123 antibody drug conjugate and PARP inhibitor are administered to said subject sequentially.

Preferably, the cancer cells are hematological cancer cells.

Preferably, said administering is carried out in vivo.

### 6. EXAMPLES

### 6.1 Example 1: Synergistic Anti-Leukemic Activity of PARP Inhibition Combined with IMGN632, an Anti-CD123 Antibody-Drug Conjugate, in Acute Myeloid Leukemia Models

Background

CD123 (IL-3 receptor alpha-chain) is a therapeutic target for hematological malignancies based on high expression levels in acute myeloid leukemia (AML), blastic plasmacytoid dendritic cell neoplasm (BPDCN), and other cancers. The anti-CD123 antibody-drug conjugate (ADC), IMGN632, comprises a humanized monoclonal antibody covalently linked to a DNA-alkylating cytotoxic payload which is currently in phase 1 evaluation for relapsed/refractory CD123-positive hematological malignancies (NCT03386513). Thus new approaches to enhance the efficacy of ADCs are of significant therapeutic interest. Poly ADP Ribose (PARP) inhibitor, olaparib, is known to synergistically enhance the activity of the CD33-targeted ADC, IMGN779, in preclinical AML models (Portwood S et al, ASH 2016).

This example demonstrates that PARP inhibition synergizes with the DNA damaging mechanism of IMGN632. The ability of olaparib and other PARP inhibitors (PARPi) in clinical development (talazoparib, niraparib, rucaparib, and veliparib) was investigated and shown to enhance the therapeutic efficacy of IMGN632 across diverse human AML cell lines and primary relapsed/refractory AML samples.

Materials and Methods

CD123 expression on human AML cell lines (HEL, HL60, MV411, Molm13, EOL-1, THP-1, and Kasumi-1) was quantified by flow cytometry using QuantriBrite beads. AML cells were continuously cultured for 72-96 hours with varying doses of IMGN632 (range 100 pM-100 nM) and specific PARP inhibitors (range 100 pM-15 μM) alone and in combination. Cell viability was measured using a WST-8 colorimetric assay. Primary clinically annotated CD123+AML cells from patients with relapsed/refractory disease were obtained under IRB-approved protocols from the Roswell Park (Buffalo, N.Y.) Hematologic Procurement Shared Resource and cultured short-term in the presence of multiple cytokines plus IMGN632+/−PARP inhibitors. Apoptosis (Annexin V/PI), cell cycle, and DNA damage (H2AX) were evaluated by flow cytometry. Additive vs. synergistic effects were determined by combination indices using Compusyn software. PARP trapping was evaluated by Western blot analysis in nuclear lysates obtained from IMGN632+/−PARP inhibitors treated AML cells.

Results

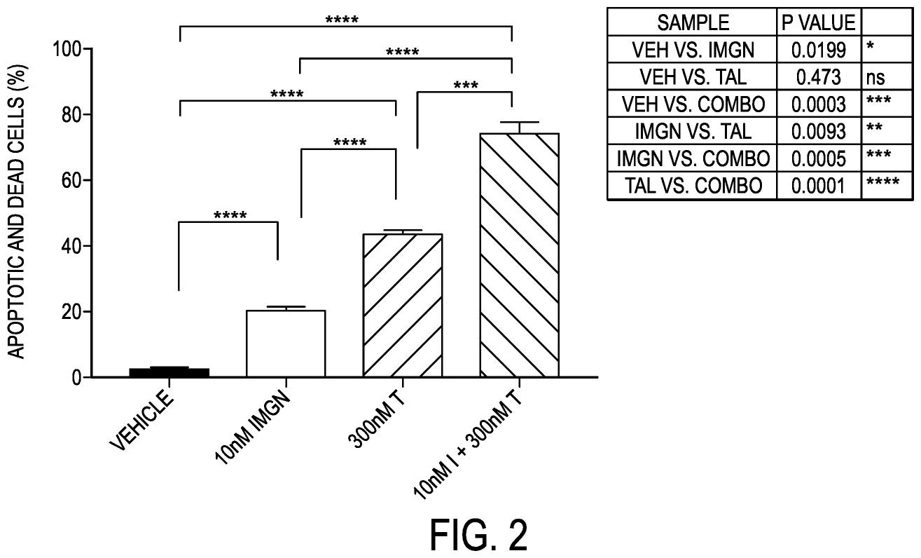

High expression levels of CD123 (range 937-2231 CD123 molecules/cell) were detected on multiple human AML cell lines (HEL-luc, MV411, Molm13, EOL-1, and THP-1) relative to unstained negative controls. Western blot analysis of nuclear lysates from AML cells demonstrated that all PARP inhibitors had varying degrees of PARP trapping on DNA. Continuous single agent 5-day treatment with all tested PARP inhibitors resulted in dose dependent in vitro inhibition of AML cell line growth with IC₅₀ values ranging from 360 nM (talazoparib, most potent) to 78 uM (veliparib, least potent). Combination therapy using PARP inhibitors (doses ranging from 300 nM-15 uM) and IMGN632 (10 nM) consistently resulted in enhanced anti-leukemic effects over monotherapy (see, e.g., FIG. 2). Synergistic anti-proliferative effects were obtained across all tested AML cell lines (n=5) with combination indexes ranging from 0.3-0.7 by Compusyn analysis. Combination therapy correlated with enhanced DNA damage, tumor cell apoptosis, and cell cycle arrest of AML cells. Moreover, IMGN632 and PARPi (olaparib or talazoparib) resulted in single agent activity against clinically annotated primary relapsed/refractory AML patient samples with evidence of synergistic effects when combined in vitro.

### CONCLUSIONS

Addition of PARP inhibitors to IMGN632, an anti-CD123 antibody-drug conjugate, further enhances DNA damage effects and consistently results in synergistic in vitro anti-leukemic effects across multiple CD123+AML cell lines and primary AML patient samples. Further studies investigating this combinatorial approach in specific molecular subtypes of AML with variable baseline sensitivities to PARPi are currently ongoing. These results indicate that PARPi is a class of agents that significantly enhance the efficacy of DNA-alkylating ADCs and/or cytotoxic chemotherapy for hematological malignancies.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

The citation of any publication is for its disclosure prior to the filing date and should not be construed as an admission that the present invention is not entitled to antedate such publication by virtue of prior invention.

What is claimed is:

1. A pharmaceutical composition comprising:
an anti-CD123 antibody-drug conjugate; and
a poly ADP ribose (PARP) inhibitor.

2. The pharmaceutical composition of claim 1, wherein the PARP inhibitor is a PARP-1 inhibitor.

3. The pharmaceutical composition of claim 1, wherein the PARP inhibitor is a PARP-2 inhibitor.

4. The pharmaceutical composition of claim 1, wherein the PARP inhibitor is selected from the group consisting of: 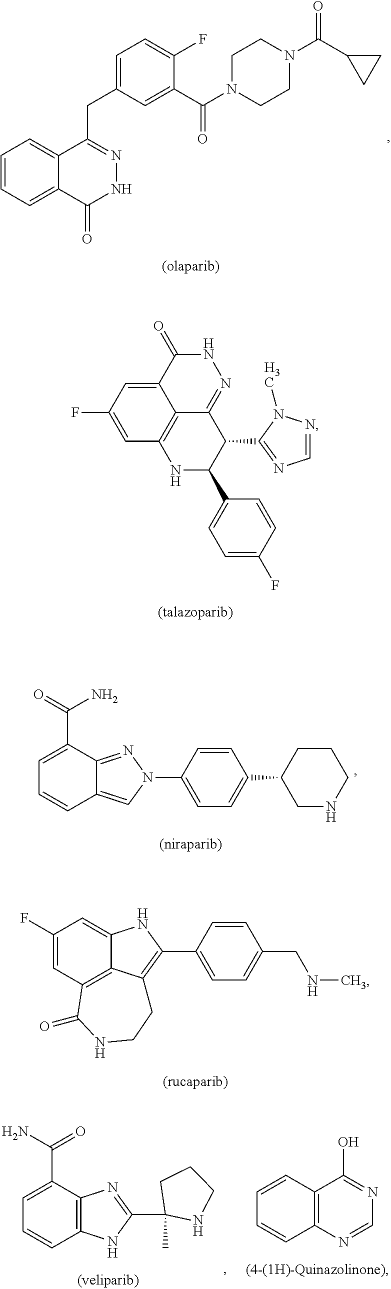 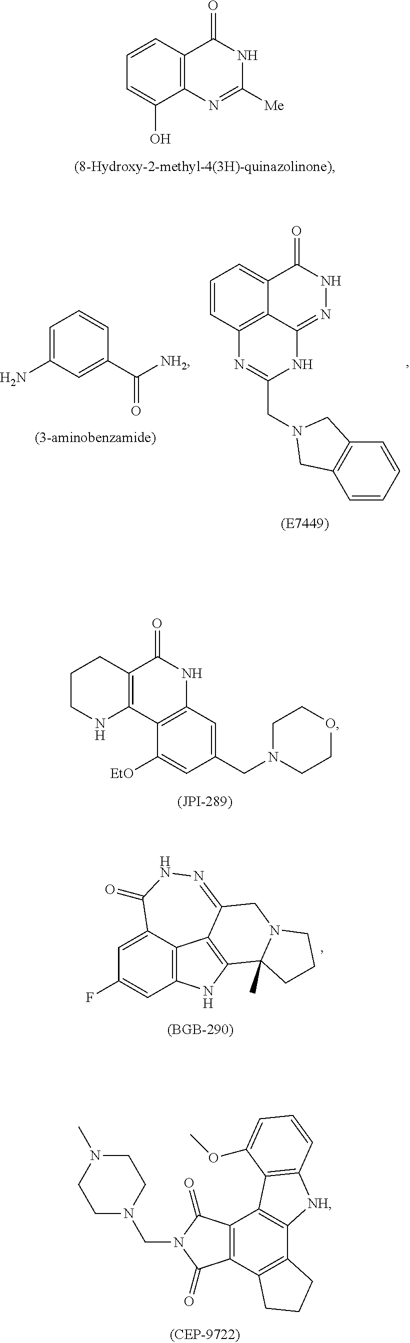 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4] 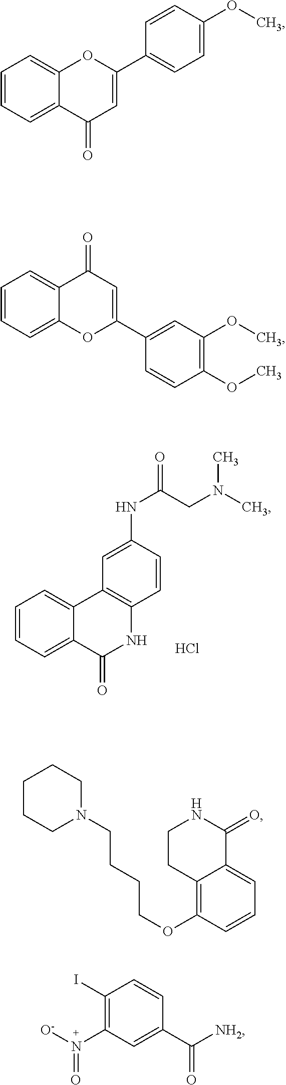
and a pharmaceutically acceptable salt thereof.

5. The pharmaceutical composition of claim 4, wherein the PARP inhibitor is olaparib or a pharmaceutically acceptable salt thereof.

6. The pharmaceutical composition of claim 4, wherein the PARP inhibitor is talazoparib or a pharmaceutically acceptable salt thereof.

7. The pharmaceutical composition of claim 1, wherein the anti-CD123 antibody-drug conjugate is an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, or an anti-CD123 antibody linked to camptothecin.

8. The pharmaceutical composition of claim 7, wherein the indolinobenzodiazepine pseudodimer is IMGN632.

9. A method for treating cancer in a subject, the method comprising administering to the subject an anti-CD123 antibody-drug conjugate and a poly ADP ribose (PARP) inhibitor in an amount effective to treat the cancer.

10. The method of claim 9, wherein the PARP inhibitor is a PARP-1 inhibitor.

11. The method of claim 9, wherein the PARP inhibitor is a PARP-2 inhibitor.

12. The method of claim 9, wherein the PARP inhibitor is selected from the group consisting of: 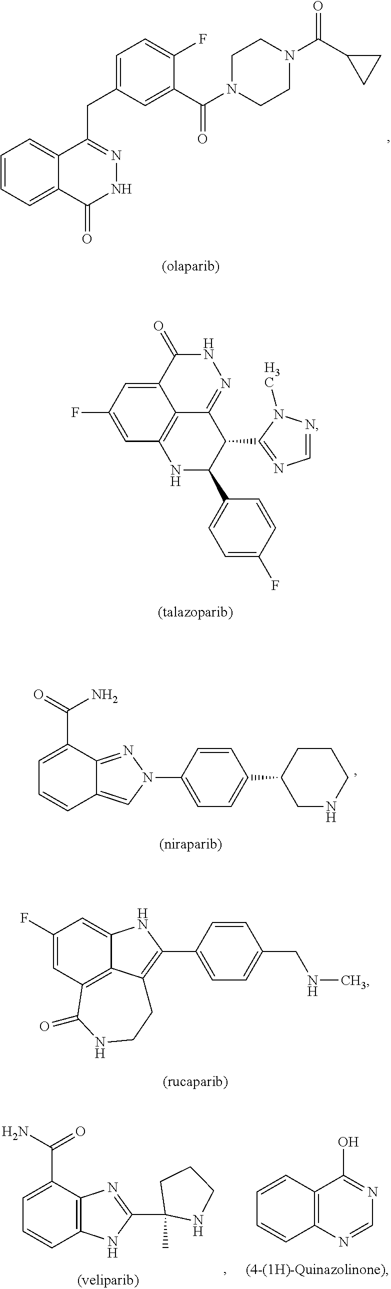 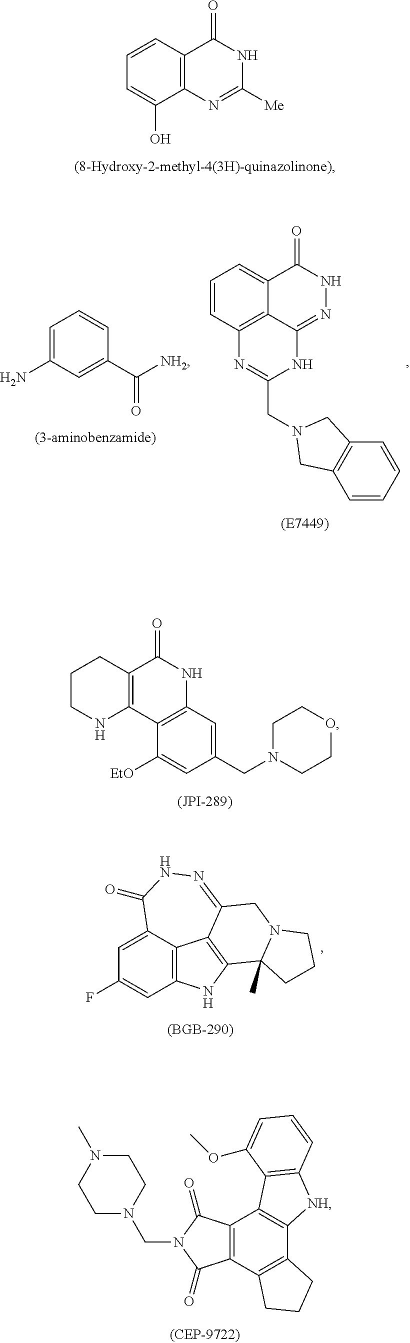 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4] 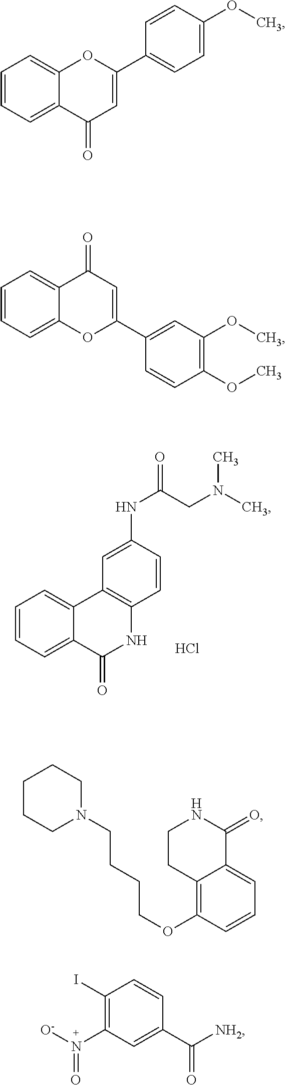
and a pharmaceutically acceptable salt thereof.

13. The method of claim 12, wherein the PARP inhibitor is olaparib or a pharmaceutically acceptable salt thereof.

14. The method of claim 12, wherein the PARP inhibitor is talazoparib or a pharmaceutically acceptable salt thereof.

15. The method of claim 9, wherein the anti-CD123 antibody-drug conjugate is selected from the group consisting of an anti-CD123 antibody linked to a indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

16. The method of claim 15, wherein the anti-CD123 anti-drug conjugate is the anti-CD123 antibody linked to indolinobenzodiazepine pseudodimer IMGN632.

17. The method of claim 9, wherein the cancer is a hematological cancer.

18. The method of claim 17, wherein the hematological cancer is selected from the group consisting of leukemia, lymphoma, and myeloma.

19. The method of claim 17, wherein the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, Hodgkin's leukemia (HL), and myeloproliferative neoplasm (MPN).

20. The method of claim 19, wherein the hematological cancer is acute myeloid leukemia.

21. The method of claim 20, wherein the acute myeloid leukemia is refractory acute myeloid leukemia.

22. The method of claim 20, wherein the acute myeloid leukemia is relapse acute myeloid leukemia.

23. The method of claim 9, wherein the subject is human.

24. The method of claim 9, wherein the anti-CD123 antibody drug conjugate and PARP inhibitor are administered to the subject simultaneously.

25. The method of claim 9, wherein the anti-CD123 antibody drug conjugate and PARP inhibitor are administered to the subject sequentially.

26. A method for inducing cancer cell death, the method comprising:
administering, to a population of cancer cells, an anti-CD123 antibody-drug conjugate and a poly ADP ribose (PARP) inhibitor in an amount effective to induce cancer cell death.

27. The method of claim 26, wherein the PARP inhibitor is a PARP-1 inhibitor.

28. The method of claim 26, wherein the PARP inhibitor is a PARP-2 inhibitor.

29. The method of claim 26, wherein the PARP inhibitor is selected from the group consisting of: 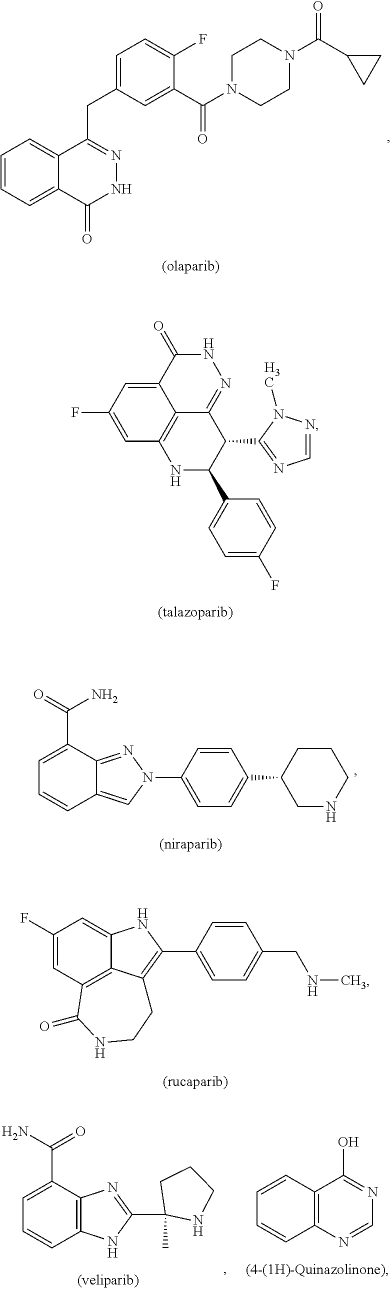 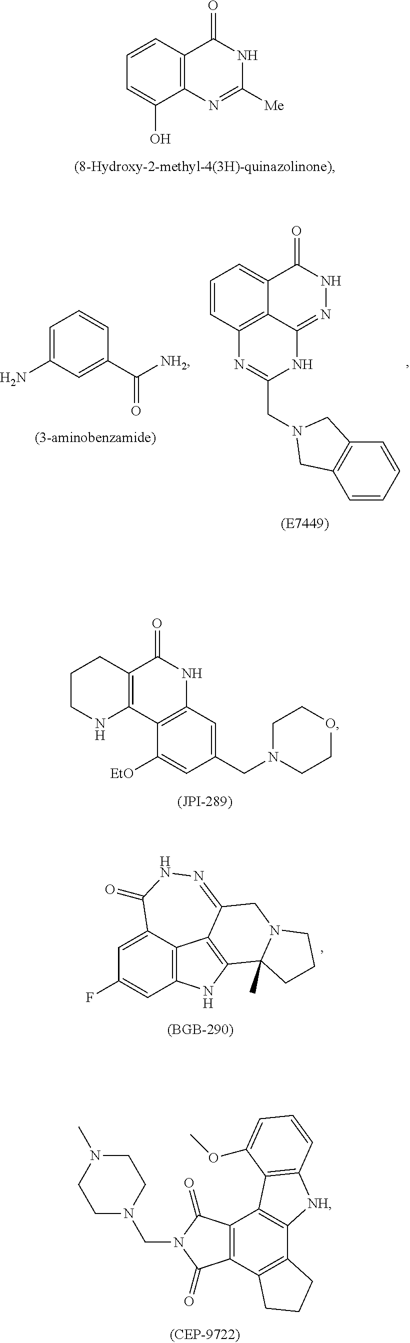 [SMILES: Cc1nc2c(O)cccc2c(=O)[nH]1 ; NC(=O)c1cccc(N)c1 ; O=c1[nH]nc2c3c(cccc13)N=C(CN1Cc3ccccc3C1)N2 ; CCOc1cc(CN2CCOCC2)cc2[nH]c(=O)c3c(c12)NCCC3 ; C[C@]12CCCN1CC1=NNC(=O)c3cc(F)cc4[nH]c2c1c34 ; COc1cccc2[nH]c3c4c(c5c(c3c12)C(=O)N(CN1CCN(C)CC1)C5=O)CCC4] 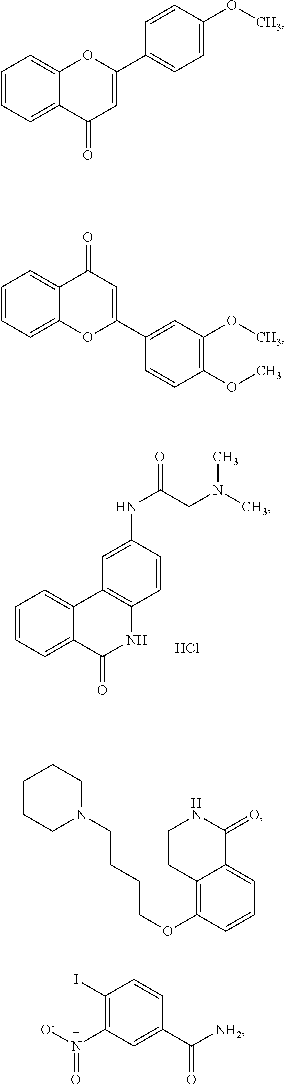
and pharmaceutically acceptable salts thereof.

30. The method of claim 29, wherein the PARP inhibitor is olaparib or a pharmaceutically acceptable salt thereof.

31. The method of claim 29, wherein the PARP inhibitor is talazoparib or a pharmaceutically acceptable salt thereof.

32. The method of claim 26, wherein the anti-CD123 antibody-drug conjugate is selected from the group consisting of an anti-CD123 antibody linked to indolinobenzodiazepine pseudodimer, an anti-CD123 antibody linked to a pyrrolobenzodiazepine dimer, an anti-CD123 antibody linked to cyclopropylpyrroloindoline, and an anti-CD123 antibody linked to camptothecin.

33. The method of claim 32, wherein the anti-CD123 anti-drug conjugate is the anti-CD123 antibody linked to indolinobenzodiazepine pseudodimer IMGN632.

34. The method of claim 26, wherein the cancer cells are hematological cancer cells.

35. The method of claim 34, wherein the hematological cancer is selected from the group consisting of leukemia, lymphoma, and myeloma.

36. The method of claim 34, wherein the hematological cancer is selected from the group consisting of acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), blastic plasmacytoid DC neoplasm (BPDCN) leukemia, non-Hodgkin lymphomas (NHL), mantle cell lymphoma, Hodgkin's leukemia (HL), and myeloproliferative neoplasm (MPN).

37. The method of claim 34, wherein the hematological cancer cells are acute myeloid leukemia cells.

38. The method of claim 37, wherein the acute myeloid leukemia cells are refractory acute myeloid leukemia cells.

39. The method of claim 37, wherein the acute myeloid leukemia cells are relapse acute myeloid leukemia cells.

40. The method of claim 26, wherein the administering is carried out in vivo.

41. A composition comprising:
an anti-CD123 antibody-drug conjugate, and
a poly ADP ribose (PARP) inhibitor
for use in therapy and/or as a medicament.

42. An anti-CD123 antibody-drug conjugate and a poly ADP ribose (PARP) inhibitor for use in treating cancer.

43. An anti-CD123 antibody-drug conjugate and a poly ADP ribose (PARP) inhibitor for use in inducing cancer cell death in a population of cancer cells.
